(19) United States
(12) Patent Application Publication
(10) Pub. No.: US 2025/0375443 A1
(43) Pub. Date: Dec. 11, 2025

(54) IDENTIFICATION AND USE OF SHP2 CONFORMATIONAL DISRUPTORS FOR CANCER TREATMENT

(71) Applicants: Xing Wang, St. Louis, MO (US); David L. Wang, Wildwood, MO (US)

(72) Inventors: Xing Wang, St. Louis, MO (US); David L. Wang, Wildwood, MO (US)

(21) Appl. No.: 19/189,964

(22) Filed: Apr. 25, 2025

Related U.S. Application Data
(60) Provisional application No. 63/658,512, filed on Jun. 11, 2024.

(51) Int. Cl. A61K 31/4965; A61K 31/58; A61K 31/155; A61K 31/409; A61K 31/7135; A61K 38/31; G01N 33/543

(52) U.S. Cl. CPC A61K 31/4965; A61K 31/58; A61K 31/155; A61K 31/409; A61K 31/7135; A61K 38/31; G01N 33/54366

(57) ABSTRACT

Compounds were identified from SHP2 Protein Conformational Array (PCA) ELISA. These compounds can disrupt SHP2 Higher Order Structure (HOS) in vitro and induce SHP2 degradation in multiple cancer cell lines. The SHP2-KRAS-ERK pathway analysis indicated that these identified compounds could induce inactivation of the active form of ERK, P-ERK. In addition, some of the compounds can induce SHP2 and/or KRAS degradation in multiple cancer cell lines. Cancer cell line testing demonstrated that these compounds identified through SHP2 HOS disruption showed dose-dependent growth inhibition. Since the SHP2-KRAS-ERK pathway plays an important role in cancer development, these compounds can be used as potential cancer treatments.

### CROSS-REFERENCE TO RELATED APPLICATION

This application claims the benefit of priority of U.S. provisional application number 63/658,512, filed June 11, 2024, the contents of which are herein incorporated by reference.

### BACKGROUND OF THE INVENTION

Currently, about 85% of protein targets are considered undruggable with the traditional drug discovery approach. Many novel strategies are being developed to overcome these and other limitations and expand the druggable protein universe.

New drug discovery platforms include small interfering RNA (siRNA), mRNA, CRISPR, PROTAC and molecular glues. Currently, drug candidates identified from these platforms are being tested in the clinic, and some of the molecules have already made it to market.

The innovation of the proposed project includes the following aspects:

1.) Protein Conformational Array (PCA) is potentially a novel drug discovery platform that uses a different mode of action in its drug candidate discovery. The PCA technology was initially developed for biologics development, including biosimilar Higher Order Structure comparability analysis (see U.S. Patent No. 10,180,434 B2, issued to Array Bridge Inc. with Xing Wang as the inventor, January 15, 2019). This has been used in novel biologics as well as biosimilar development for a decade with data accumulated demonstrating its sensitivity, specificity and systematic coverage.

Basically, overlapping peptides were chemically synthesized to cover the target protein whole sequence, each peptide was conjugated to KLH and immunize rabbits to produce polyclonal antibodies toward the peptide linear epitopes. Previous studies have shown that it only takes 3-6 amino acid residues to form one linear epitope, therefore the typical 30-amino acid peptide in theory can produce multiple linear epitope antibodies.

The distinct groups of antibodies have shown good specificity in recognizing their corresponding peptides, thus providing information for the specific regions where the epitopes have been changed. The microplate-based ELISA is measuring the linear epitope exposure on the surface of the target protein; by quantifying the amount of linear epitope exposure and distribution, one can deduce the Higher Order structure status of the protein, and earlier studies have shown that this assay can detect HOS change from any physical or chemical perturbations such as temperature, pH, oxidation and aggregation.

2.) The PCA platform can target any protein target but especially more valuable the undruggable targets such as SHP2. SHP2 is a protein phosphatase that plays a significant role in cancer development. The development of effective inhibitors targeting the SHP2 phosphatase enzyme active site has not been successful because of specificity issues.

In recent years, a novel approach has emerged for the identification of allosteric regulators to circumvent the specificity issue to disrupt SHP2 function. Potent allosteric regulators have been identified and demonstrate good antitumor activity in animal tumor models and some of them have been advanced to clinical trials.

### SUMMARY OF THE INVENTION

The present invention is directed to compositions, methods, and systems for identifying and utilizing compounds that modulate the higher order structure (HOS) of proteins, particularly the SHP2 protein, which plays a critical role in oncogenic signaling pathways. In particular, the invention relates to a novel use of Protein Conformational Array (PCA) technology and associated ELISA-based assays for the identification of therapeutic compounds that disrupt SHP2 conformational integrity, thereby altering its activity and associated downstream signaling.

In one aspect, the invention provides a list of fifteen compounds identified through PCA-based screening that disrupt the conformational structure of SHP2. These compounds were further validated through direct ELISA, sandwich ELISA, and cell-based assays, and several of them exhibit anti-tumor activity, SHP2 degradation, and suppression of ERK signaling. Importantly, five of the compounds are FDA-approved drugs previously used for non-oncology indications, demonstrating the potential for drug repurposing.

In another aspect, the invention provides derivative compounds based on the fifteen lead structures by the addition of one to five chemical groups, thereby expanding the chemical space of SHP2-targeting agents while retaining or enhancing their bioactivity. These derivatives may be synthesized and evaluated using the same conformational and cellular assays disclosed herein.

The invention further encompasses a suite of screening and analytical tools, including a direct ELISA platform and sandwich ELISA method, both of which enable the detection of HOS disruption by monitoring epitope exposure across the protein structure. Additionally, a method of whole-molecule PCA analysis is provided to elucidate the mode of action of test compounds by profiling antibody binding across multiple conformational epitopes. These assays can be applied to SHP2 or adapted for use with other protein targets.

In yet another aspect, the invention includes a method for monitoring compound efficacy in cancer cells using Western blot analysis of SHP2, KRAS, P-ERK, and β-Actin, which serves as a marker for the compound’s modulation of the SHP2-KRAS-ERK signaling axis. These tools enable comprehensive assessment of both molecular and phenotypic effects of candidate compounds.

Accordingly, the present invention provides new therapeutic compositions and methods for cancer treatment and offers a powerful platform for identifying conformational disruptors of proteins that have traditionally been classified as undruggable.

These and other features, aspects and advantages of the present invention will become better understood with reference to the following drawings, description and claims.

### BRIEF DESCRIPTION OF THE DRAWINGS

FIG. 1 depicts is a list of compounds for SHP2 conformational disruption;

FIG. 2 depicts charts of SHP2 sandwich ELISA hit confirmation;

FIG. 3 depicts charts of the top-10 shp2 sandwich ELISA confirmation - whole molecule profile;

FIG. 4 depicts charts of the direct ELISA confirmation full profile;

FIG. 5 depicts schematic views of cell line H1197 screening identified compounds inactivating the ERK pathway or SHP2 degradation;

FIG. 6 depicts schematic views of cell line a549 screening identified compound inactive the ERK pathway;

FIG. 7 depicts charts of cell line PC-3 testing of FDA-approved drugs; and

FIG. 8 depicts charts of cell line PC-3 testing of NCI Compounds.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As stated above, the present invention is directed towards the identified compounds, which not only showed dose-dependent Higher Order Structure disruption but also demonstrated in vivo SHP2 degradation which has never been reported for SHP2 allosteric regulators.

In an extension application of the PCA technology, we developed three PCA assays for SHP2, KRAS and p53 respectively. To our knowledge, this is a unique platform focusing on the identification of protein conformational disruption in drug discovery. Both the NCI Diversity Compound Library and a commercial FDA-approved drug library were screened. All the NCI Diversity Library compounds were associated with previous work on the NCI-60 cancer cell line studies including GI50, LC50, TGI50 and dose-dependent cancer cell inhibition but none was known to target SHP2, thus our findings provide new insight for these identified compounds.

The SHP2 PCA platform identified 15 compounds disrupting SHP2 including 5 FDA-approved drugs, indicating this platform is not selecting compounds detrimental to human cells in general. For the FDA-approved drugs, several drugs with initially targeting non-cancer disease were identified. This result is encouraging in principle because it showed that compounds identified with the PCA technology could pass clinical testing and make it to the market.

In our cancer cell line studies, we selected 6 cancer cell lines with SHP2 gene up-regulation as selection standard and tested 5 FDA-approved drugs and 10 best NCI Diversity Library hits. In addition to the inhibition of cell viability, we also demonstrated that some of the FDA-approved drugs and Diversity library hits can inhibit the SHP2-KRAS-ERK pathway by inactivating P-ERK. Furthermore, a small group of compounds including the best NCI Diversity Library compound identified in the 6 cancer cell line testing can induce SHP2 and/or KRAS degradation. This observation is the first for SHP2-targeting compounds including both phosphatase inhibitors and allosteric regulators identified so far.

The identification of compounds disrupting SHP2 Higher Order Structure will allow the development of treatments for cancer. The compounds have shown the disruption of antitumor target SHP2 in vitro and activities in multiple cancer cell lines.

The claimed invention differs from what currently exists. Targeting SHP2 Higher Order Structure stability is a unique approach, which is based in part on our previous patent in Protein Conformational Array. We are the only party using this approach for antitumor compound screening.

Previous approaches for SHP2 allosteric regulators were screened based on phosphatase activity which limited the scope of compounds that could be identified.

The screening using SHP2 Protein Conformational Array could select compounds acting in three separate modes of action instead of just activity inhibition.

Also, the invention can be used for disease treatment in CNS and septic shock treatments.

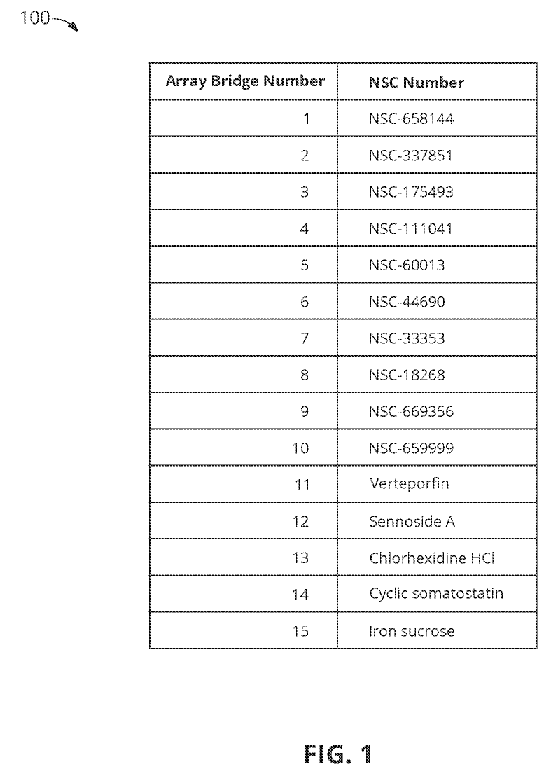

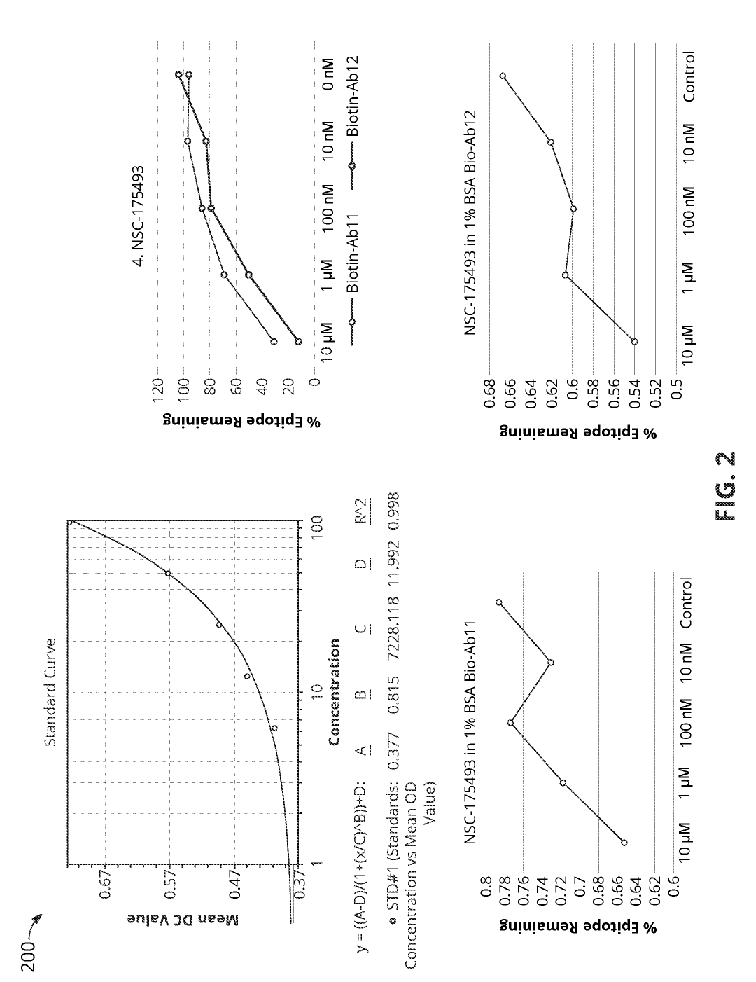

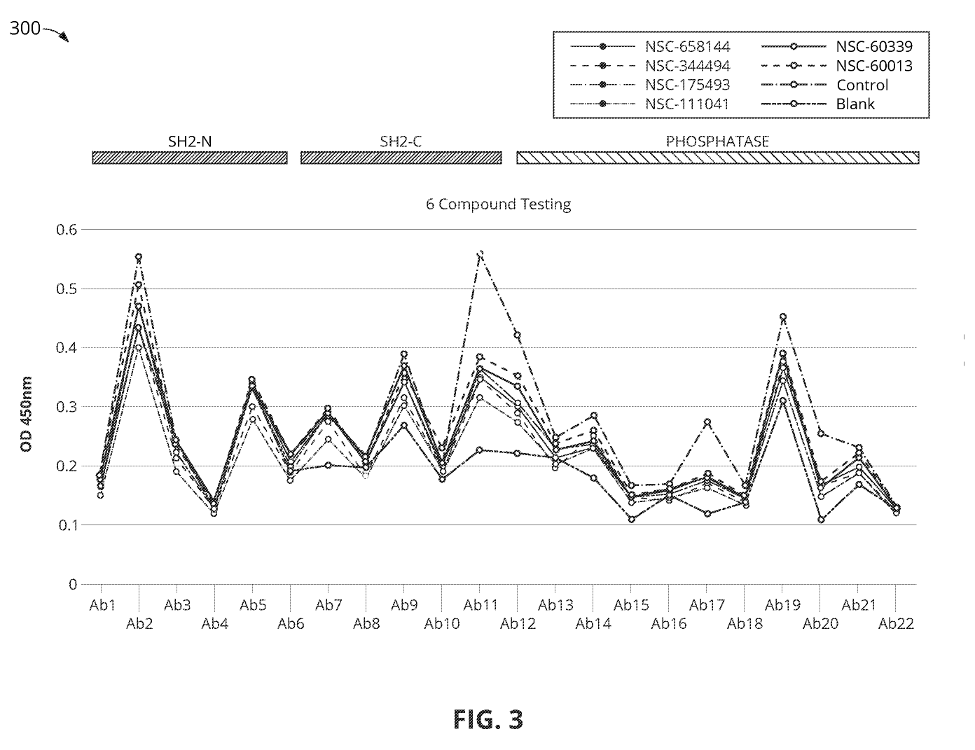

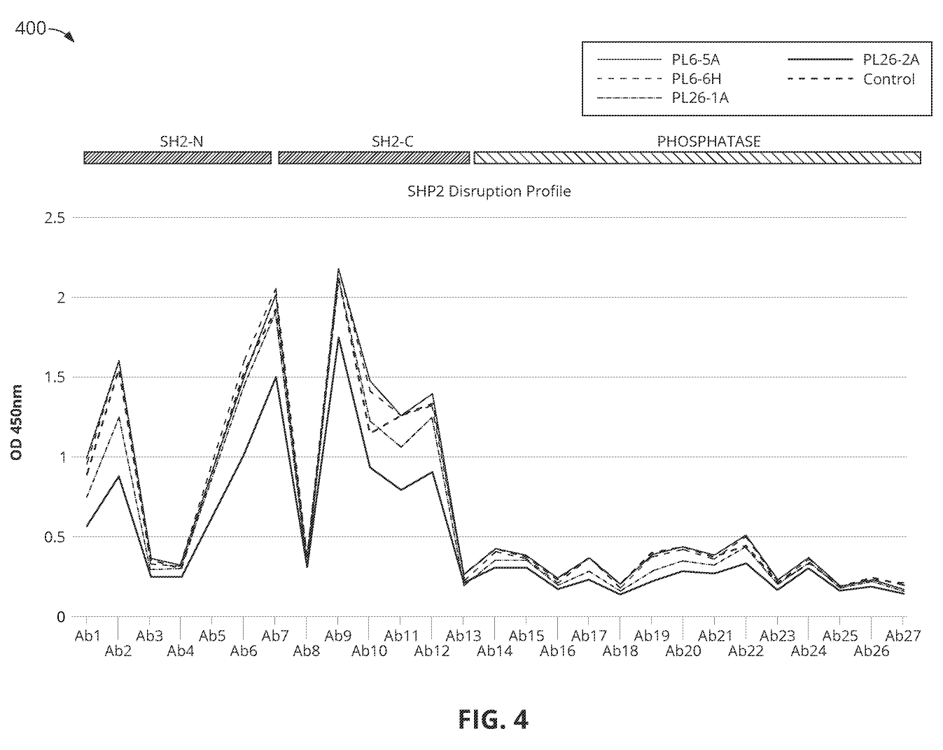

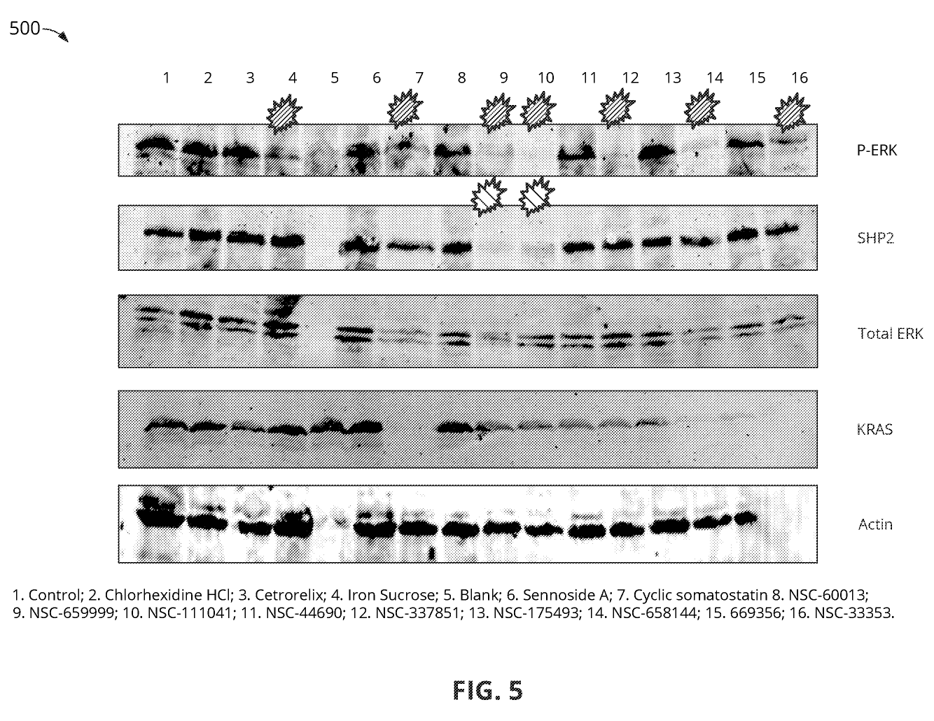

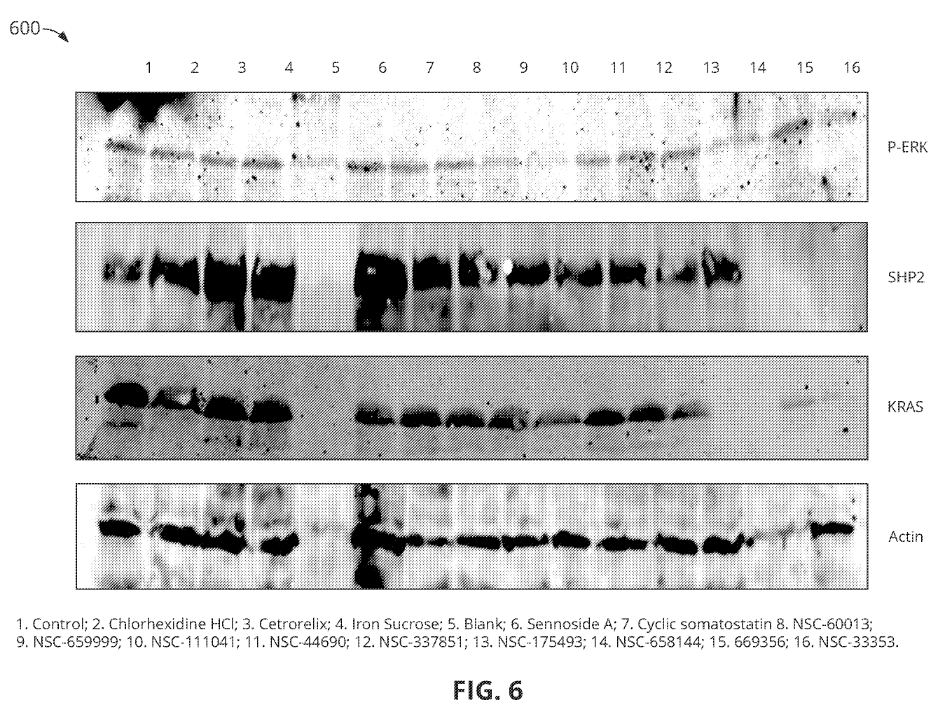

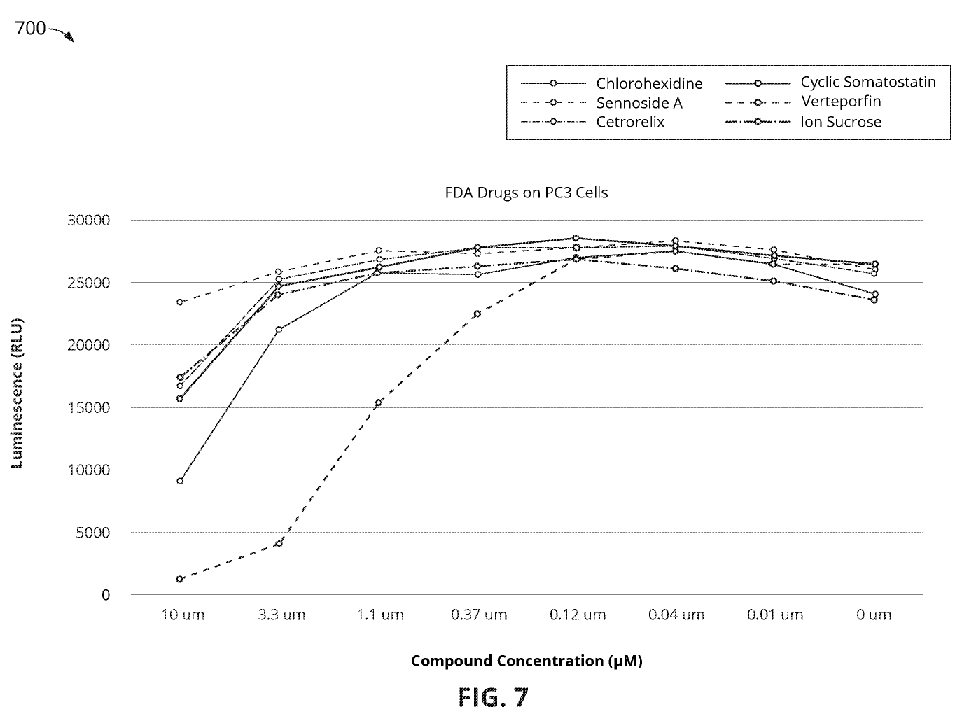

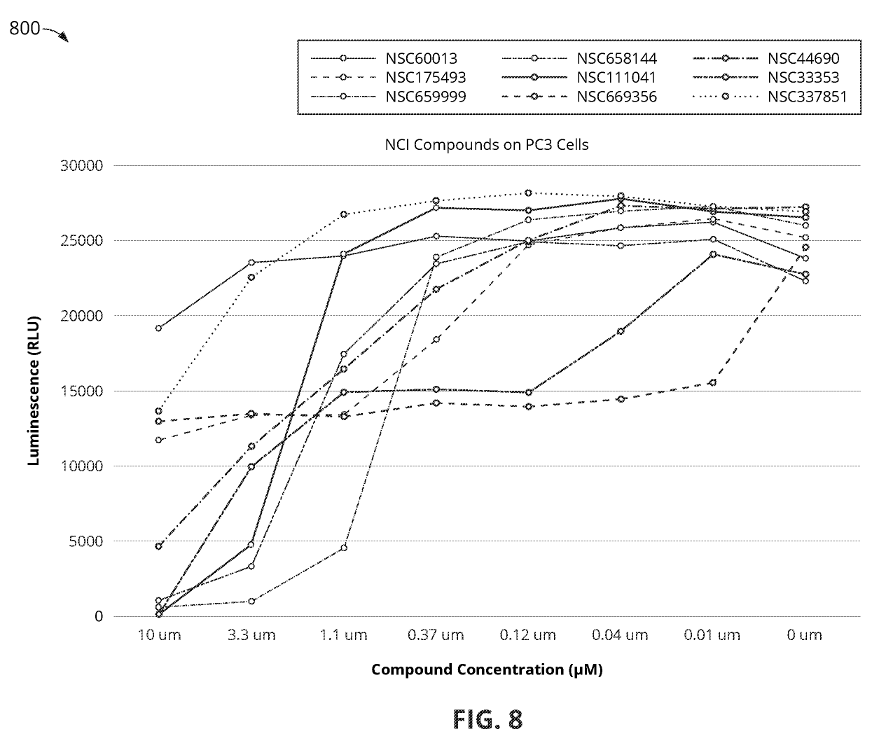

As discussed herein, and as seen by way of example in FIGS. 1-8 hereto, in a preferred embodiment the present invention comprises:

1. Compounds identified by the SHP2 screening.

2. SHP2 Sandwich ELISA

3. SHP2 Direct ELISA

4. Cancer cell lines

5. SHP2 whole profile analysis

6. Western Blot for SHP2, KRAS, ERK, β-Actin

### Relationship Between The Components:

SHP2 sandwich ELISA, whole profile ELISA and cancer cell lines were used for the identification and validation of the compounds.

### How The Invention Works:

The compounds identified can be used as monotherapy or in combination with other anti-cancer drugs as combo therapy for cancer treatments.

### How To Make The Invention:

Through chemical synthesis.

Animal tumor model and human clinical trials will further validate the identified compounds as anti-tumor agents.

The compounds identified can be used as the starting points to identify more potent and/or specific compounds for SHP2 disruption.

### How To Use The Invention:

The invention can be used as monotherapy or in combination with other marketed drugs as combo therapy for cancer treatment.

Additionally, these compounds can also be used for disease treatment in other therapeutic areas such as CNS.

Also, the invention can be used for disease treatment in CNS and septic shock treatments.

It should be understood, of course, that the foregoing relates to exemplary embodiments of the invention and that modifications may be made without departing from the spirit and scope of the invention as set forth in the following claims.

What is claimed is:

1. A composition comprising at least one compound selected from a group of fifteen compounds as listed in Figure 1 and identified by their ability to disrupt the higher order structure (HOS) of SHP2 protein.

2. The composition of claim 1, wherein at least one compound exhibits anti-tumor activity in SHP2-overexpressing cancer cell lines.

3. A compound derived from a compound of claim 1, wherein the derivative compound includes the addition of one to five chemical groups selected from the group consisting of alkyl, aryl, amino, hydroxyl, halogen, and carboxyl functional moieties.

4. The compound of claim 3, wherein the derivative retains the ability to disrupt SHP2 conformational structure as determined by an epitope-based ELISA assay.

5. A system for identifying molecules capable of disrupting protein higher order structure (HOS), comprising:
(a) a direct ELISA assay platform configured to detect epitope exposure on a target protein;
(b) a plurality of test compounds;
(c) a readout mechanism configured to detect conformational changes based on signal changes in epitope-specific binding.

6. The system of claim 5, wherein the target protein is SHP2.

7. A method for evaluating the ability of a compound to disrupt the higher order structure of a target protein, comprising:
(a) providing a sandwich ELISA comprising antibodies specific to linear epitopes of the target protein;
(b) contacting the target protein with a test compound;
(c) measuring signal changes to assess epitope exposure and determine disruption of the target protein HOS.

8. The method of claim 7, wherein the target protein is SHP2.
